(19) United States
(12) Patent Application Publication
(10) Pub. No.: US 2008/0167290 A1
(43) Pub. Date: Jul. 10, 2008

(54) Combination of a Serotonin Reuptake Inhibitor and Amoxapine

(75) Inventors: Thomas Ivo Cremers, Groningen (NL); Sandra Willigers, Bagsvaerd (DK); Jorn Arnt, Solrod Strand (DK)

Correspondence Address: DARBY & DARBY P.C., P.O. BOX 770, Church Street Station, New York, NY, 10008-0770, US

(73) Assignee: H. Lundbeck A/S, Valby-Copenhagen (DK)

(21) Appl. No.: 10/569,346

(22) PCT Filed: Sep. 1, 2004
(86) PCT No.: PCT/DK04/00580

§ 371 Date: Jul. 12, 2006

Related U.S. Application Data
(60) Provisional application No. 60/500,792, filed on Sep. 4, 2003.

(30) Foreign Application Priority Data
Sep. 4, 2003 (DK) PA 2003 01269

(51) Int. Cl. A61K 31/553; A61P 25/00

(52) U.S. Cl. 514/211.13

(57) ABSTRACT

The present invention relates to the use of a combination of Amoxapine and a serotonin reuptake inhibitor (SRI), or any other compound, which causes an elevation in the level of extracellular serotonin, for the treatment of depression and other affective disorders.

The present invention relates to the use of a combination of Amoxapine and a serotonin reuptake inhibitor (SRI), or any other compound, which causes an elevation in the level of extracellular serotonin, for the treatment of depression and other affective disorders.

### BACKGROUND

Selective serotonin reuptake inhibitors (hereinafter referred to as SSRIs) have become first choice therapeutics in the treatment of depression, certain forms of anxiety and social phobias, because they are effective, well tolerated and have a favourable safety profile compared to the classic tricyclic antidepressants.

However, clinical studies on depression and anxiety disorders indicate that non-response to SSRIs is substantial, up to 30%. Another, often neglected, factor in antidepressant treatment is compliance, which has a rather profound effect on the patient's motivation to continue pharnacotherapy.

First of all, there is the delay in therapeutic effect of SSRIs. Sometimes symptoms even worsen during the first weeks of treatment. Secondly, sexual dysfunction is a side effect common to all SSRIs. Without addressing these problems, real progress in the pharmacotherapy of depression and anxiety disorders is not likely to happen.

In order to cope with non-response, psychiatrists sometimes make use of augmentation strategies. Augmentation of antidepressant therapy may be accomplished through the co-administration of mood stabilizers such as lithium carbonate or triiodothyronin or by the use of electroshock.

In 1993, an augmentation strategy with pindolol was described by Artigas et al. in Trends Pharmacol. Sci. 1993, 14, p 262-263. Artigas' idea is based on intracerebral microdialysis experiments in animals. In fact, later neurochemical studies built on the desensitization hypothesis by Blier and co-workers stated that the delay in therapeutic effect of antidepressants is related to a gradual desensitization of 5-HT autoreceptors (Blier et al. J. Clin. Psycipharmacol. 1987, 7 suppl. 6, 24S-35S). A key point in their hypothesis is that the effects of SSRIs on the release-controlling somatodendritic autoreceptors (5-HT_{1A}) limit the release of 5-HT in terminal areas and thus the effect of 5-HT uptake inhibition in those regions. This is supported by microdialysis experiments in rats, showing that the increase in extracellular 5-HT elicited by a single dose of an SSRI is augmented by co-administration of a 5-HT_{1A} autoreceptor antagonist (Invernizzi et al. Brain Res, 1992, 584, p 322-324 and Hjorth, S., J. Neurochem, 1993, 60, p 776-779).

The effect of combined administration of a compound that inhibits serotonin reuptake and a 5-HT_{1A} receptor antagonist has been evaluated in several studies (Innis, R. B. et al. Eur. J. Pharmacol. 1987, 143, p. 1095-204 and Gartside, S. E., Br. J. Pharmacol, 1995, 115, p 1064-1070, Blier, P. et al. Trends in Pharmacol. Science 1994, 15, 220). In these studies it was found that 5-HT_{1A} receptor antagonists would abolish the initial brake on 5-HT neurotransmission induced by the serotonin reuptake inhibitors and thus produce an immediate boost of 5-HT transmission and a rapid onset of therapeutic action.

Several patent applications have been filed which cover the use of a combination of a 5-HT_{1A} antagonist and a serotonin reuptake inhibitor for the treatment of depression (see e.g. EP-A2-687 472 and EP-A2-714 663).

Another approach to increase terminal 5-HT would be through blockade of the 5-HT_{1B} autoreceptor. Microdialysis experiments in rats have indeed shown that increase of hippocampal 5-HT by citalopram is potentiated by GMC 2-29, an experimental 5-HT_{1B} receptor antagonist.

Several patent applications covering the combination of an SSRI and a 5-HT_{1B} antagonist or partial agonist have also been filed (WO 97/28141, WO 96/03400, EP-A-701819 and WO 99/13877).

### SUMMARY OF THE INVENTION

It has now surprisingly been found that Amoxapine or pharmaceutically acceptable salts thereof may be used to augment and provide faster onset of the therapeutic effect of serotonin reuptake inhibitors.

In one aspect the invention relates to use of Amoxapine or a pharmaceutically acceptable salt thereof for the preparation of a pharmaceutical composition to be used in combination with a serotonin reuptake inhibitor or any other compound, which causes an elevation in the level of extracellular serotonin.

In another aspect the invention relates to use of Amoxapine or a pharmaceutically acceptable salt thereof for the preparation of a pharmaceutical composition useful for augmenting and/or providing faster onset of the therapeutic effect of a serotonin reuptake inhibitor or any other compound, which causes an elevation in the level of extracellular serotonin.

In a further aspect the invention relates to a pharmaceutical composition or kit comprising Amoxapine or a pharmaceutically acceptable salt thereof and a compound, which is a serotonin reuptake inhibitor, or any other compound which causes an elevation in the level of extracellular serotonin, and optionally pharmaceutically acceptable carriers or diluents.

In a further aspect the invention relates to a method for the treatment of diseases or disorders responsive to a serotonin reuptake inhibitor or any other compound which causes an elevation in the level of extracellular serotonin, comprising administering Amoxapine or a pharmaceutically acceptable salt thereof and a serotonin reuptake inhibitor, or a compound which causes an elevation in the level of extracellular serotonin, to an individual in need thereof.

In a further aspect the invention relates to use of Amoxapine or a pharmaceutically acceptable salt thereof and a compound, which is a serotonin reuptake inhibitor, or any other compound which causes an elevation in the level of extracellular serotonin, for the preparation of a pharmaceutical composition for the treatment of diseases or disorders responsive to the therapeutic effect of a serotonin reuptake inhibitor or any other compound causing an elevation in the level of extracellular serotonin.

In a further aspect the invention relates to the use of Amoxapine or a pharmaceutically acceptable salt thereof and a compound, which is a serotonin reuptake inhibitor, or any other compound which causes an elevation in the level of extracellular serotonin, for the preparation of a kit for the treatment of diseases or disorders responsive to the therapeutic effect of a serotonin reuptake inhibitor or any other compound causing an elevation in the level of extracellular serotonin.

In a further aspect the invention relates to a method for augmenting and/or providing faster onset of the therapeutic effect of a serotonin reuptake inhibitor, or any other compound which causes an elevation in the level of extracellular serotonin, comprising administering Amoxapine or a pharmaceutically acceptable salt thereof to an individual to be treated with or undergoing treatment with the serotonin reuptake inhibitor, or any other compound which causes an elevation in the level of extracellular serotonin.

In an embodiment the serotonin reuptake inhibitor or the compound which causes an elevation in the level of extracellular serotonin is used in the treatment of depression, anxiety disorders and other affective disorders, eating disorders such as bulimia, anorexia and obesity, phobias, dysthymia, premenstrual syndrome, cognitive disorders, impulse control disorders, attention deficit hyperactivity disorder and drug abuse, in particular depression.

In a further embodiment the serotonin reuptake inhibitor or the compound which causes an elevation in the level of extracellular serotonin is used in the treatment of anxiety disorders including general anxiety disorder, panic anxiety, obsessive compulsive disorder, acute stress disorder, post trauma stress disorder or social anxiety disorder.

In a further embodiment, the serotonin reuptake inhibitor or the compound which causes an elevation in the level of extracellular serotonin is used in the treatment of psychoses, including schizophrenia and schizoaffective disorders.

In a further embodiment the serotonin reuptake inhibitor is selected from citalopram, escitalopram, fluoxetine, sertraline, paroxetine, fluvoxamine, venlafaxine, dapoxetine, nefazodone, imipramin, femoxetine and clomipramine or a pharmaceutically acceptable salt of any of these compounds. Just to clarify, each of the serotonin reuptake inhibitors specified above is intended to be an individual embodiment. Accordingly, each of them and the use thereof may be claimed individually.

In a further preferred embodiment, the serotonin reuptake inhibitor is escitalopram.

In a further preferred embodiment, the serotonin reuptake inhibitor is citalopram.

In a further embodiment the serotonin reuptake inhibitor is a selective serotonin reuptake inhibitor (SSRI).

In a further embodiment the pharmaceutical composition or kit prepared is adapted for simultaneous administration of the active ingredients. In an embodiment the active ingredients are contained in the same unit dosage form.

In a further embodiment the pharmaceutical composition or kit prepared is adapted for sequential administration of the active ingredients. In an embodiment the active ingredients are contained in discrete unit dosage forms.

### DESCRIPTION OF THE INVENTION

The present invention relates to the use of Amoxapine or a pharmaceutically acceptable salt thereof for the preparation of a pharmaceutical composition to be used in combination with a serotonin reuptake inhibitor or any other compound, which causes an elevation in the level of extracellular serotonin.

In particular, the present invention relates to the use of Amoxapine or a pharmaceutically acceptable salt thereof for the preparation of a pharmaceutical composition useful for augmenting and/or providing faster onset of the therapeutic effect of a serotonin reuptake inhibitor or any other compound, which causes an elevation in the level of extracellular serotonin.

More particularly, the present invention relates to the use as above, of Amoxapine, or a pharmaceutically acceptable salt thereof, for the treatment of depression, anxiety disorders and other affective disorders, such as generalized anxiety disorder, panic anxiety, obsessive compulsive disorder, acute stress disorder, post traumatic stress disorder and social anxiety disorder eating disorders such as bulimia, anorexia and obesity, phobias, dysthymia, premenstrual syndrome, cognitive disorders, impulse control disorders, attention deficit hyperactivity disorder, psychosis including schizophrenia and schizoaffective disorders and drug abuse, in particular depression, in combination with a serotonin reuptake inhibitor or any other compound, which causes an elevation in the level of extracellular serotonin.

The anxiety disorders mentioned above include general anxiety disorder, panic anxiety, obsessive compulsive disorder, acute stress disorder, post trauma stress disorder or social anxiety disorder.

As used herein, the term augmenting covers improving the therapeutic effect and/or potentiating the therapeutic effect of an SRI or a compound which causes an elevation in the level of extracellular 5-HT.

In a further embodiment, the invention relates to the use of Amoxapine or a pharmaceutically acceptable salt thereof and a compound, which is a serotonin reuptake inhibitor, or a compound, which causes an elevation in the level of extracellular serotonin, for the preparation of a pharmaceutical composition for the treatment of diseases or disorders responsive to the therapeutic effect of a serotonin reuptake inhibitor, or any other compound, which causes an elevation in the level of extracellular serotonin.

In a further embodiment, the invention relates to the use of Amoxapine or a pharmaceutically acceptable salt thereof and a compound, which is a serotonin reuptake inhibitor, or a compound, which causes an elevation in the level of extracellular serotonin, for the preparation of a kit-of-parts (kit) for the treatment of diseases or disorders responsive to the therapeutic effect of a serotonin reuptake inhibitor, or any other compound, which causes an elevation in the level of extracellular serotonin.

The diseases responsive to a serotonin reuptake inhibitor include depression, anxiety disorders and other affective disorders, eating disorders such as bulimia, anorexia and obesity, phobias, dysthymia, premenstrual syndrome, cognitive disorders, impulse control disorders, attention deficit hyperactivity disorder, psychosis, including schizophrenia and schizoaffective disorders, psychosis, including schizophrenia and schizoaffective disorders and drug abuse, in particular depression.

The term anxiety disorders is as defined above.

In one embodiment, the present invention relates to the use of Amoxapine or a pharmaceutically acceptable salt thereof for the preparation of a pharmaceutical composition as above, which is adapted for simultaneous administration of the active ingredients. In particular, such pharmaceutical compositions may contain the active ingredients within the same unit dosage form, e.g. in the same tablet or capsule. Such unit dosage forms may contain the active ingredients as a homogenous mixture or in separate compartments of the unit dosage form.

In another embodiment, the present invention relates to the use of Amoxapine or a pharmaceutically acceptable salt thereof for the preparation of a pharmaceutical composition or kit as above, which is adapted for sequential administration of the active ingredients. In particular, such pharmaceutical compositions may contain the active ingredients in discrete unit dosage forms, e.g. discrete tablets or capsules containing either of the active ingredients. These discrete unit dosage forms may be contained in the same container or package, e.g. a blister pack.

As used herein the term kit means a pharmaceutical composition containing each of the active ingredients, but in discrete unit dosage forms.

The invention also relates to a pharmaceutical composition or kit comprising Amoxapine or a pharmaceutically acceptable salt thereof and a compound, which is a serotonin reuptake inhibitor, or any other compound, which causes an elevation in extracellular 5-HT and optionally pharmaceutically acceptable carriers or diluents.

The pharmaceutical composition or kit of the invention may be adapted for simultaneous administration of the active ingredients or for sequential administration of the active ingredients, as described above.

Finally, the present invention relates to a method for the treatment of diseases or disorders responsive to a serotonin reuptake inhibitor or any other compound, which causes an elevation in the level of extracellular serotonin, comprising administering Amoxapine or a pharmaceutically acceptable salt thereof and a serotonin reuptake inhibitor, or a compound, which causes an elevation in the level of extracellular serotonin, to an individual in need thereof.

In particular, the present invention relates to a method for augmenting and/or providing faster onset of the therapeutic effect of a serotonin reuptake inhibitor or any other compound, which causes an elevation in the level extracellular serotonin, comprising administering Amoxapine or a pharmaceutically acceptable salt thereof to an individual to be treated with or undergoing treatment with the serotonin reuptake inhibitor or any other compound, which causes an elevation in the level of extracellular serotonin. The individuals, which may benefit from treatment with a combination as above, may suffer from depression, anxiety disorders and other affective disorders, psychosis, eating disorders such as bulimia, anorexia and obesity, phobias, premenstrual syndrome, dysthymia, cognitive disorders, impulse control disorders, attention deficit hyperactivity disorder, psychosis, and drug abuse, in particular depression.

As mentioned above, anxiety disorder includes general anxiety disorder, panic anxiety, obsessive compulsive disorder, acute stress disorder, post trauma stress disorder or social anxiety disorder.

Psychosis includes but is not limited to schizophrenia and schizoaffective disorders.

Amoxapine and the serotonin reuptake inhibitor may be administered simultaneously as described above.

Alternatively, the active ingredients may be administered sequentially, e.g. in two discrete unit dosage forms as described above.

It has now, surprisingly, been found that co-administration of Amoxapine and a serotonin reuptake inhibitor produces a significant increased response in an animal model predictive of antidepressant effect, the 5-HT microdialysis model, compared to the administration of the serotonin reuptake inhibitor alone. Administration of Amoxapine alone causes no significant effect in the experiments.

As mentioned above, serotonin reuptake inhibitors show delayed onset of action. Even in responders to SSRIs, several weeks of treatment are necessary to achieve a relief in symptoms. Amoxapine may provide fast onset of therapeutic effect of serotonin reuptake inhibitors.

The use of a combination of Amoxapine and a serotonin reuptake inhibitor may greatly reduce the amount of serotonin reuptake inhibitor necessary to treat depression and other affective disorders and may thus reduce the side effects caused by the serotonin reuptake inhibitor. In particular, the combination of a reduced amount of SRI and Amoxapine may reduce the risk of SSRI-induced sexual dysfunction and sleep disturbances.

Co-administration of Amoxapine and a serotonin reuptake inhibitor may also be useful for the treatment of refractory depression, i.e. depression, which cannot be treated appropriately by administration of a serotonin reuptake inhibitor alone. Typically, Amoxapine may be used as add-on therapy for the augmentation of the response to SRIs in patients where at least 40-60% reduction in symptoms has not been achieved during the first 6 weeks of treatment with an SRI.

Many antidepressants with serotonin reuptake inhibiting effect have been described in the literature. Any pharmacologically active compound which primarily or partly exerts its therapeutic effect via inhibition of serotonin reuptake in the CNS, may benefit from augmentation with Amoxapine.

The following list contains a number of serotonin reuptake inhibitors, which may benefit from augmentation with Amoxapine: citalopram, escitalopram, fluoxetine, R-fluoxetine, sertraline, paroxetine, fluvoxamine, venlafaxine, duloxetine, dapoxetine, nefazodone, imipramine, imiprarnine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran, bazinaprine, YM 922, S 33005, F 98214-TA, OPC 14523, alaproclate, cyanodothepine, trimipramine, quinupramine, dothiepin, Amoxapine, nitroxazepine, McN 5652, McN 5707, O177, Org 6582, Org 6997, Org 6906, amitriptyline, amitriptyline N-oxide, nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine and clovoxamine. The compounds mentioned above may be used in the form of the base or a pharmaceutically acceptable acid addition salt thereof. Each of the serotonin reuptake inhibitors specified above is intended to be an individual embodiment. Accordingly, each of them and the use thereof may be claimed individually.

Typically, compounds such as citalopram, escitalopram, fluoxetine, R-fluoxetine, sertraline, paroxetine, fluvoxamine, venlafaxine, desmethylvenlafaxine, duloxetine, dapoxetine, vilazodone, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, imeldine, ifoxetine, indeloxazine, tiflucarbine, viqualine, milnacipran, bazinaprine, alaproclate, cyanodothepine, trimipramine, quinupramine, dothiepin, Amoxapine, nitroxazepine, roxindole, amitriptyline, amitriptyline N-oxide, nortriptyline, pirlindole, indatraline, napamezole, diclofensine, trazodone, sercloremine, nitroquipazine, ademethionine, sibutramine, desmethylsubitramine, didesmethylsubitramine, clovoxamine vilazodone,

N-[(1-[(6-Fluoro-2-naphthalenyl)methyl]-4-piperidinyl]amino]carbonyl]-3-pyridine carboxamide (WY 27587),
[trans-6-(2-chlorophenyl)-1,2,3,5,6,10b-hexahydropyrrolo-(2,1-a)isoquinoline] (McN 5707),
(dl-4-exo-amino-8-chloro-benzo-(b)-bicyclo[3.3.1]nona-2-6 alpha(10 alpha)-diene hydrochloride)(Org 6997),
(dl)-(5 alpha,8 alpha,9 alpha)-5,8,9,10-Tetrahydro-5,9-methanobenzocycloocten-8-amine hydrochloride (Org 6906),
-[2-[4-(6-fluoro-1H-indol-3-yl)-3,6-dihydro-1(2H)-pyridinyl]ethyl]-3-isopropyl-6-(methylsulphonyl)-3,4-dihydro-1H-2,1,3-benzothiadiazine-2,2-dioxide (LY393558), [4-(5,6-dimethyl-2-benzofuranyl)-piperidine] (CGP 6085),
dimethyl-[5-(4-nitro-phenoxy)-6,7,8,9-tetrahydro-5H-benzocyclohepten-7-yl]-amine (RU 25.591),

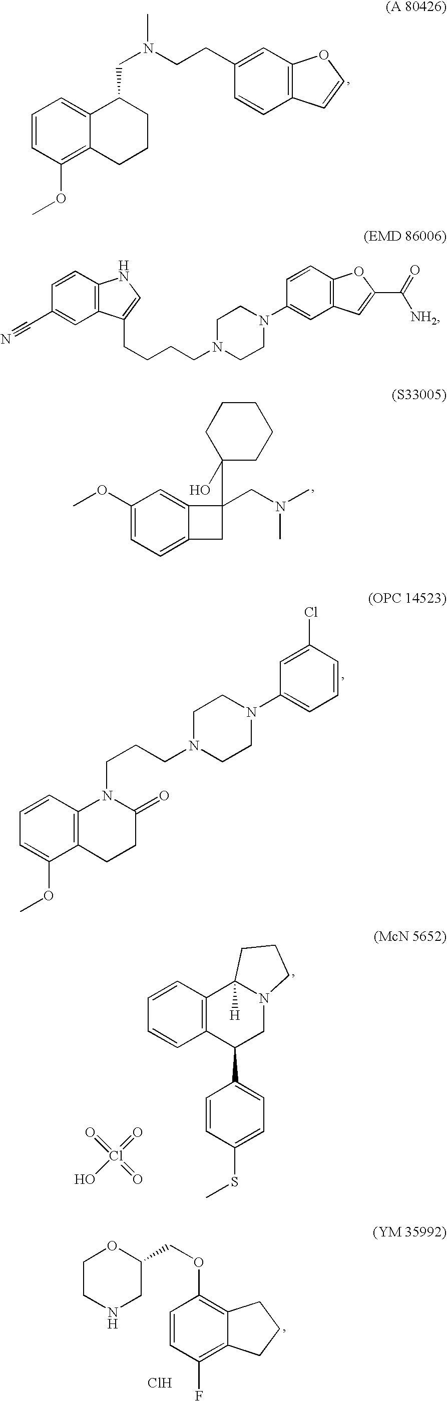

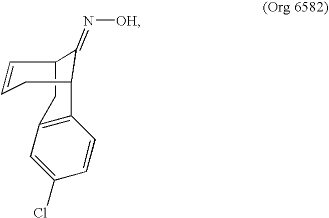

are suitable as SRIs. The compounds mentioned above may be used in the form of the base or a pharmaceutically acceptable acid addition salt thereof. Each of the serotonin reuptake inhibitors specified above is intended to be an individual embodiment. Accordingly, each of them and the use thereof may be claimed individually.

Other therapeutic compounds which may benefit from augmentation with Amoxapine include compounds, which causes an elevation in the extracellular level of 5-HT in the synaptic cleft, although they are not serotonin reuptake inhibitors. One such compound is tianeptine.

The above list of serotonin reuptake inhibitors and other compounds, which causes an increase in the extracellular level of serotonin, may not be construed as limiting.

In an embodiment the SRIs is selected from citalopram, escitalopram, fluoxetine, sertraline, paroxetine, fluvoxamine, venlafaxine, dapoxetine, nefazodone, imipramin, femoxetine and clomipramine. Just to clarify, each of these SRIs constitute individual embodiments, and may be the subject of individual claims.

The term selective serotonin reuptake inhibitor (SSRI) means an inhibitor of the monoamnine transporters which has stronger inhibitory effect at the serotonin transporter than the dopamine and the noradrenaline transporters.

Selective serotonin reuptake inhibitors (SSRIs) are among the most preferred serotonin reuptake inhibitors used according to the present invention. Thus in a further embodiment the SRI is selected from SSRIs, such as citalopram, escitalopram, fluoxetine, fluvoxamine, sertraline or paroxetine.

The active ingredients according to the invention, i.e. Amoxapine and the SRI or a compound causing an increase in extracellular serotonin levels, may be used in the free base form or in the form of a pharmaceutically acceptable acid addition salt thereof, the latter being obtainable by reaction of the base form with an appropriate acid.

Citalopram is preferably used in the form of the hydrobromide or as the base, escitalopram in the form of the oxalate, fluoxetine, sertraline and paroxetine in the form of the hydrochloride and fluvoxamine in the form of the maleate.

As mentioned above, the combination of Amoxapine with a serotonin reuptake inhibitor unexpectedly shows a synergistic effect on the central nervous system (CNS). As a consequence, combination therapy using Amoxapine and lower doses of the serotonin reuptake inhibitor than normally used in monotherapy, may be effective, and side-effects associated with the larger amounts of serotonin reuptake inhibitor used in monotherapy may be reduced or prevented altogether.

Additionally, combination therapy with Amoxapine using a normal dose of serotonin reuptake inhibitor has the advantage that an effective CNS effect may be obtained in the often large number of patients who do not respond to conventional monotherapy with SSRIs.

The amount of Amoxapine used in combination therapy may range from about 0.001 to about 1 g/day, such as from about 0.001 to about 0.1 mg/day, about 0.1 to about 1 mg/day, about 1 mg/day to about 10 mg/day, about 10 mg/day to about 100 mg/day and from about 100 mg/day to about 1 g/day.

Serotonin reuptake inhibitors, including the SSRIs specifically mentioned hereinabove, differ both in molecular weight and in activity. As a consequence, the amount of serotonin reuptake inhibitor used in combination therapy depends on the nature of said serotonin reuptake inhibitor. In one embodiment of the invention, the serotonin reuptake inhibitor or the compound causing an increase in the level of extracellular 5-HT, is administered at lower doses than required when the compound is used alone. In another embodiment, the serotonin reuptake inhibitor or the compound causing an increase in the level of extracellular 5-HT, is administered in normal doses.

To prepare the pharmaceutical compositions of this invention, an appropriate amount of the active ingredient(s), in salt form or base form, is combined in an intimate admixture with a pharmaceutically acceptable carrier, which can take a wide variety of forms depending on the form of preparation desired for administration. These pharmaceutical compositions are desirably in unitary dosage form suitable for administration orally, rectally, percutaneously or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed.

It is especially advantageous to formulate the aforementioned pharmaceutical compositions in dosage unit form for ease of administration and uniformity of dosage. As used in the specification and claims, unit dosage form refers to physically discrete units suitable as unitary dosages, each unit containing a predetermined quantity of active ingredient(s) calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Amoxapine may be administered before, during or after the administration of the serotonin reuptake inhibitor provided that the time between the administration of Amoxapine and the administration of the serotonin reuptake inhibitor is such that ingredients are allowed to act synergistically on the CNS. When simultaneous administration of Amoxapine and a serotonin reuptake inhibitor is envisaged, a composition containing both a serotonin reuptake inhibitor and Amoxapine may be particularly convenient. Alternatively, Amoxapine and the serotonin reuptake inhibitor may be administered separately in the form of suitable compositions. The compositions may be prepared as described hereinabove.

The present invention also comprises products containing Amoxapine and a serotonin reuptake inhibitor as a combination preparation for simultaneous, separate or sequential use in psychiatric drug therapy. Such products may comprise, for example, a kit comprising discrete unit dosage forms containing Amoxapine and discrete unit dosage forms containing a serotonin reuptake inhibitor, all contained in the same container or pack, e.g. a blister pack.

The above mentioned preparations for simultaneous or sequential administration may instead of a serotonin reuptake inhibitor contain another compound causing an elevation in the level of extracellular 5-HT.

### Experimental Part

### Animals

Male albino rats of a Wistar-derived strain (285-320 g; Harlan, Zeist, The Netherlands) were used for the experiments. Upon surgery, rats were housed individually in plastic cages (35×35×40 cm), and had free access to food and water. Animals were kept on a 12 h light schedule (light on 7:00 a.m.). The experiments are concordant with the declarations of Helsinki and were approved by the animal care committee of the faculty of mathematics and natural science of the University of Groningen.

### Drugs

The following drugs were used: escitalopram oxalate and amoxapine(Lundbeck A/S, Copenhagen, Denmark)

### Surgery

Microdialysis of brain serotonin levels was performed using home made I-shaped probes, made of polyacrylonitrile/sodium methyl sulfonate copolymer dialysis fiber (i.d. 220 μm, o.d. 0.31 μm, AN 69, Hospal, Italy). Preceding surgery rats were anaesthetised using isoflurane (O₂/N₂O; 300/300 ml/min). Lidocaine-HCl, 10% (m/v) was used for local anaesthesia. Rats were placed in a stereotaxic frame (Kopf, USA), and probes were inserted into Ventral Hippcampus (V. Hippo, L +4.8 mm, IA: +3.7 mm, V: −8.0 mm) (Paxinos and Watson, 1982). After insertion, probes were secured with dental cement.

### Microdialysis Experiments

Rats were allowed to recover for at least 24 h. Probes were perfused with artificial cerebrospinal fluid containing 147 mM NaCl, 3.0 mM KCl, 1.2 mM CaCl₂, and 1.2 mM MgCl₂, at a flow-rate of 1.5 μl/min (Harvard apparatus, South Natick, Mass., USA). 15 minute microdialysis samples were collected in HPLC vials containing 7.5 μl 0.02 M acetic acid for serotonin analysis.

### Serotonin Analysis:

Twenty-μl microdialysate samples were injected via an autoinjector (CMA/200 refrigerated microsampler, CMA, Sweden) onto a 100×2.0 mm C18 Hypersil 3 μm column (Bester, Amstelveen, the Netherlands) and separated with a mobile phase consisting of 5 g/L di-ammoniumsulfate, 500 mg/L EDTA, 50 mg/L heptane sulphonic acid, 4% methanol v/v, and 30 μl/L of triethylamine, pH 4.65 at a flow of 0.4 ml/min (Shimadzu LC-10 AD). 5-HT was detected amperometrically at a glassy carbon electrode at 500 mV vs Ag/AgCl (Antec Leyden, Leiden, The Netherlands). The detection limit was 0.5 fmol 5-HT per 20 μl sample (signal to noise ratio 3).

### Data Presentation and Statistics

Four consecutive microdialysis samples with less then 20% variation were taken as control and set at 100%. Data are presented as per entages of control level (mean±S.E.M.) in time. Statistical analysis was c performed using Sigmastat for Windows (SPSS, Jandel Corporation). Treatments were compared versus controls using two way analysis of variance (ANOVA) for repeated measurements, followed by Student Newman Keuls test. Drug effects were evaluated using one way ANOVA for repeated measures on ranks. Level of significance level was set at p<0.05.

### Results

Co-administration of escitalopram with amoxapine on 5-HT levels in ventral hippocampus

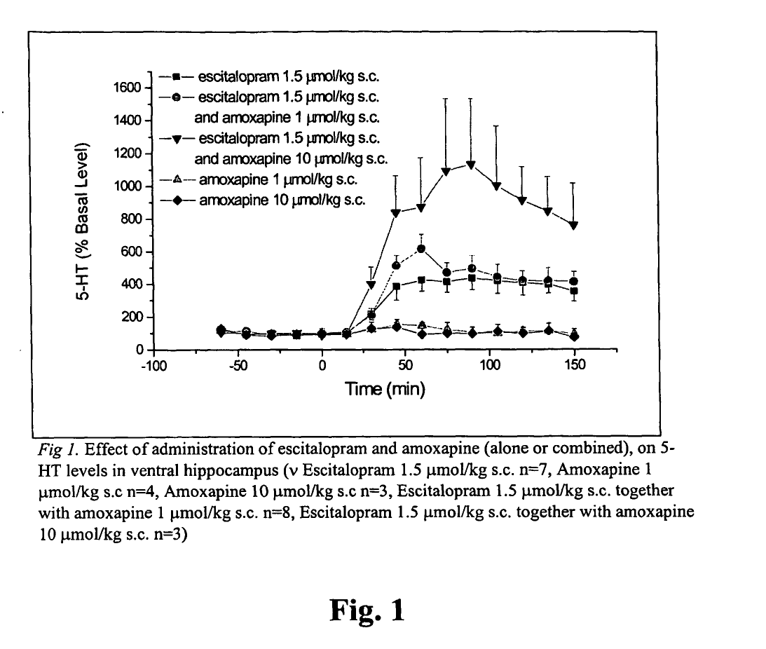

Administration of both doses of amoxapine (1 and 10 μmol/kg s.c.) did not induce any significant effects on hippocampal 5-HT levels (X²₁₆=16.6 P=0.08, X²₁₀=9.63, P=0.47). Co-administration of escitalopram (1.5 μmol/kg s.c.) with 1 μmol/kg s.c. of amoxapine did not induce an augmented effect of escitalopram on 5-HT levels (Treatment F(1,13)=0.464, P=0.50). Co-administration of 10 μmol/kg s.c. of amoxapine with 1.5 μmol/kg escitalopram significantly augmented the increase in 5-HT levels (Treatment F(1,8)=7.35, P=0.0265, Treatment versus time F(1,106)=6.34, P<0.0001). Posthoc analysis revealed significant differences at t=75 and 90 minutes (see FIG. 1).

1. (canceled)

2. A method for augmenting and/or providing faster onset of the therapeutic effect of a serotonin reuptake inhibitor or any other compound, which causes an elevation in the level of extracellular serotonin, the method comprising administering a pharmaceutical composition comprising (a) Amoxapine or a pharmaceutically acceptable salt thereof, (b) a compound, which is a serotonin reuptake inhibitor, or any other compound which causes an elevation in the level of extracellular serotonin, and (c) optionally a pharmaceutically acceptable carrier or diluent to a patient in need thereof.

3. The method according to claim 1 wherein the serotonin reuptake inhibitor or the compound which causes an elevation in the level of extracellular serotonin is administered to treat a disorder selected from depression, anxiety disorders and other affective disorders, eating disorders, phobias, dysthymia, premenstrual syndrome, cognitive disorders, impulse control disorders, attention deficit hyperactivity disorder, psychosis, and drug abuse.

4. The method according to claim 3 wherein the serotonin reuptake inhibitor or the compound which causes an elevation in the level of extracellular serotonin is used in the treatment of anxiety disorders including general anxiety disorder, panic anxiety, obsessive compulsive disorder, acute stress disorder, post trauma stress disorder or social anxiety disorder.

5. The method according to claim 3 wherein the serotonin reuptake inhibitor or the compound which causes an elevation in the level of extracellular serotonin is administered to treat depression.

6. The method according to claim 2 wherein the serotonin reuptake inhibitor is selected from citalopram, escitalopram, fluoxetine, sertraline, paroxetine, fluvoxamine, venlafaxine, dapoxetine, nefazodone, imipramin, femoxetine and clomipramine or a pharmaceutically acceptable salt of any of these compounds.

7. The method according to claim 2 wherein the serotonin reuptake inhibitor is a selective serotonin reuptake inhibitor.

8. The method according to claim 2 wherein the pharmaceutical composition prepared is adapted for simultaneous administration of the active ingredients.

9. The method according to claim 8 wherein the active ingredients are contained in the same unit dosage form.

10. The method according to claim 2 wherein the pharmaceutical composition prepared is adapted for sequential administration of the active ingredients.

11. A pharmaceutical composition comprising Amoxapine or a pharmaceutically acceptable salt thereof and a compound, which is a serotonin reuptake inhibitor, or any other compound which causes an elevation in the level of extracellular serotonin, and optionally pharmaceutically acceptable carriers or diluents.

12. The pharmaceutical composition according to claim 11 wherein the serotonin uptake inhibitor is selected from citalopram, escitalopram, fluoxetine, sertraline, paroxetine, fluvoxamine, venlafaxine, dapoxetine, nefazodone, imipramin, femoxetine and clomipramine or a pharmaceutically acceptable salt of any of these compounds.

13. The pharmaceutical composition according to claim 11 which is adapted for simultaneous administration of the active ingredients.

14. The pharmaceutical composition according to claim 13 wherein the active ingredients are contained in the same unit dosage form.

15. A kit comprising Amoxapine or a pharmaceutically acceptable salt thereof and a compound, which is a serotonin reuptake inhibitor, or any other compound which causes an elevation in the level of extracellular serotonin, and optionally pharmaceutically acceptable carriers or diluents.

16. The kit according to claim 15 wherein the serotonin uptake inhibitor is selected from citalopram, escitalopram, fluoxetine, sertraline, paroxetine, fluvoxamine, venlafaxine, dapoxetine, nefazodone, imipramin, femoxetine and clomipramine or a pharmaceutically acceptable salt of any of these compounds.

17. The kit according to claim 15 which is adapted for simultaneous administration of the active ingredients.

18. The kit according to claim 15 which is adapted for sequential administration of the active ingredients.
